(19) United States
(12) Patent Application Publication
(10) Pub. No.: US 2001/0039004 A1
(43) Pub. Date: Nov. 8, 2001

(54) Method for preventing cryoprotectant toxicity and chilling injury

(76) Inventor: Gregory M. Fahy, Corona, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/771,221

(22) Filed: Jan. 26, 2001

Related U.S. Application Data
(60) Provisional application No. 60/178,157, filed on Jan. 26, 2000.

(51) Int. Cl. C12N 5/08

(52) U.S. Cl. 435/1.3

(57) ABSTRACT

A method for cooling, rewarming, and removing cryopreservative of living cells by decreasing injury and cryotoxicity is presented. The method was developed by attempting to simulate without freezing, the events that take place during freezing living cells and/or tissue.

### RELATED APPLICATIONS

This applications claims priority of U.S. Provisional Application No. 60/178,157, filed Jan. 26, 2000.

### FIELD OF THE INVENTION

This invention relates generally to the field of cryopreservation. More specifically, the present invention relates to a method for preventing cryoprotectant toxicity and chilling injury upon cooling and warming of tissue.

### BACKGROUND OF THE INVENTION

During the cooling and warming of tissues or cells in cryopreservative, the cells and tissues are often injured. This can also occur upon removal of the cryopreservative before use of the cells or tissue.

### SUMMARY OF THE INVENTION

A method for cooling, rewarming, and removing cryopreservative of living cells by decreasing injury and cryotoxicity is presented. The method was developed by attempting to simulate without freezing, the events that take place during freezing living cells and/or tissue.

### BRIEF DESCRIPTION OF THE DRAWINGS

### DETAILED DESCRIPTION OF THE PREFERRED EMBODIMENT

A method for cooling, rewarming, and removing cryopreservative of living cells by decreasing injury and cryotoxicity is presented. The method was developed by attempting to simulate without freezing, the events that take place during freezing living cells and/or tissue.

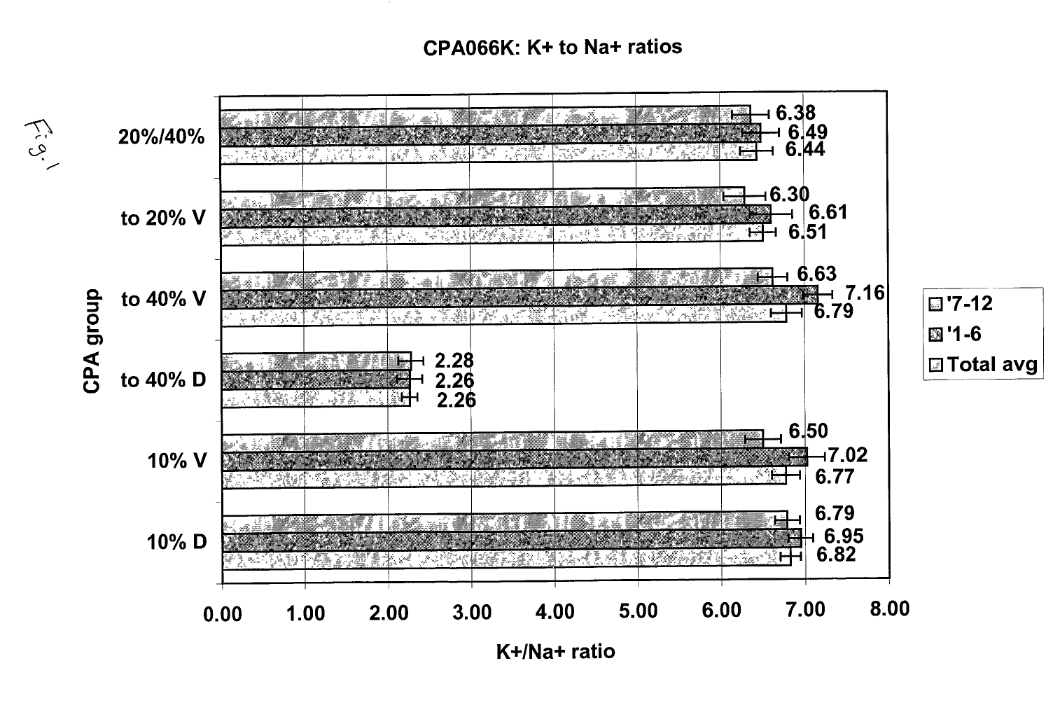

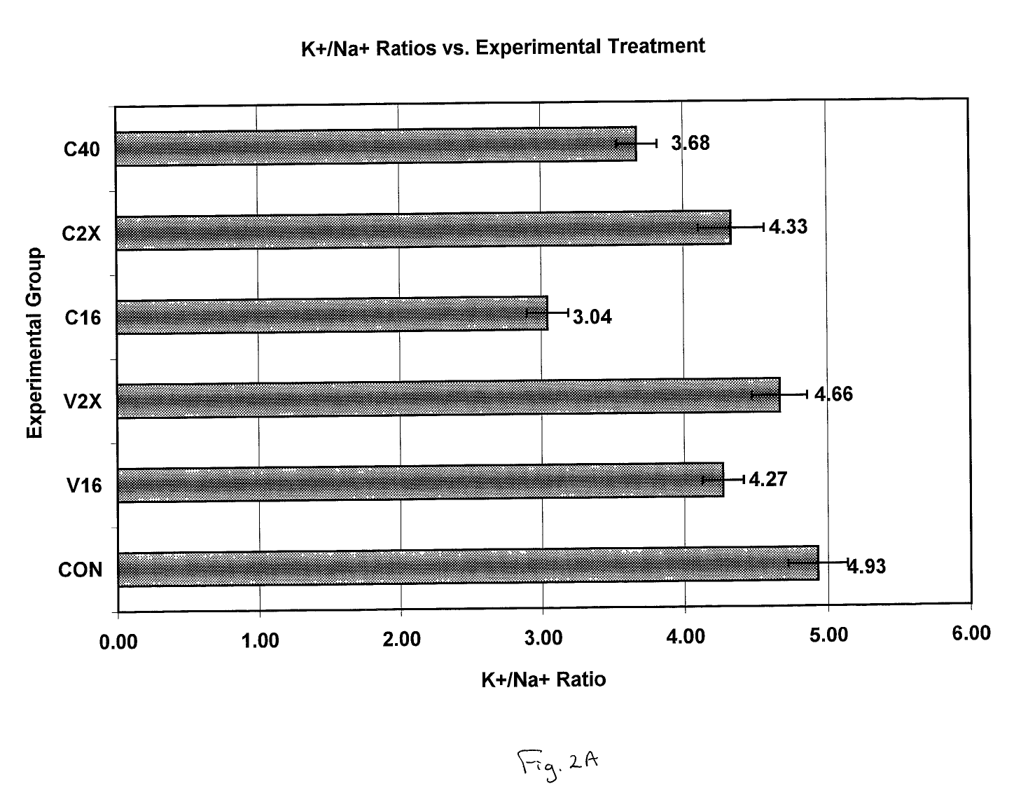

FIG. 1 shows the effects of simulating the freezing of slices in either 10% w/v DMSO or 10% w/v Veg (Veg is a mixture of DMSO, Formamide, and Ethylene Glycol in the proportion as explained in U.S. patent application Ser. No. 09/400,793 filed Sep. 21, 1999, (herein incorporated by reference). The lowest two bars are the viabilities of the slices (K/Na ratios) after exposure to 10% DMSO or to 10% Veg only. These concentrations are innocuous, and viability is 100% of that of fresh tissue slices. The data for 10% cryoprotectant (DMSO or Veg) are control data and represent what slices would experience at the time freezing begins. The second group of bars from the top of the figure is another control, showing the effects of exposing slices to 20% w/v Veg only with no simulated freezing. Again, the K/Na ratios are in the range expected for fresh, untreated tissue. The key results are the remaining three groups of bars, labeled “20%/40%”, “to 40% V”, and “to 40% D”. In the first group, “20%/40%”, the simulation assumed that slices were frozen in 20% Veg until the total concentration of Veg reached 40%, or, in other words, until the liquid volume of the solution had been reduced to one half the initial volume. In such a situation, the vehicle solution, RPS-2 (minus calcium and magnesium) would also be concentrated by a factor of two. Remarkably, this group demonstrated essentially no toxicity despite exposure to a vehicle solution concentrated by a factor of two at zero degrees C (all exposures done at 0 degrees C). The group labeled “to 40% V” is even more remarkable. In this group, the simulation was of freezing in 10% Veg to a total concentration of 40% Veg, which means that, in this case, the RPS-2 vehicle was also concentrated four fold. However, amazingly, this group continued to show no demonstrable toxicity attributed to either the Veg it self or the concentrated vehicle. The final group, “to 40% D”, was the same as the “to 40% V” group, except that the cryoprotectant was 40% w/v DMSO in 4× RPS-2 instead of 40% w/v Veg in 4× RPS-2. This group was damaged, but this is not surprising because 40% DMSO is know to be very toxic. If anything, however, the toxicity of the 40% DMSO was less than would have been expected had the DMSO been exposed to the slices in an isotonic (1×) RPS-2 vehicle (carrier) solution.

In this experiment, slices exposed to 40% DMSO or 40% Veg were diluted, after this exposure, back to the initial reference solution in one step, with no “osmotic buffer” such as mannitol added to offset the dilution of the cryoprotectant. For example, in the 20%/40% group, slices were transferred from 40% Veg in 2× RPS-2 to 20% Veg in 1× RPS-2. Slices in the “to 40% V” group were transferred from 40% Veg in 4× RPS-2 to 10% Veg in 1× RPS-2 in one step, and slices in the “to 40% D” group were transferred from 40% w/v DMSO in 4× RPS-2 to 10% DMSO in 1× RPS-2 to 10% DMSO in 1× RPS-2. After these initial dilutions, the remaining cryoprotectant was washed out in the presence of 300 mM mannitol as an osmotic buffer, in accordance with previously published methods.

In the figure, the dark bars refer to six slices treated as a separate group, and the white bars refer to six separate slices handled as though they were in an independent treatment group. As is evident from inspection of the figure, both duplicate sets of slices for each treatment were in close agreement, validating the overall average results for each group, which are provided by the grey bars.

This was experiment CPA066K. The numerical data for all groups is given in Table 1.

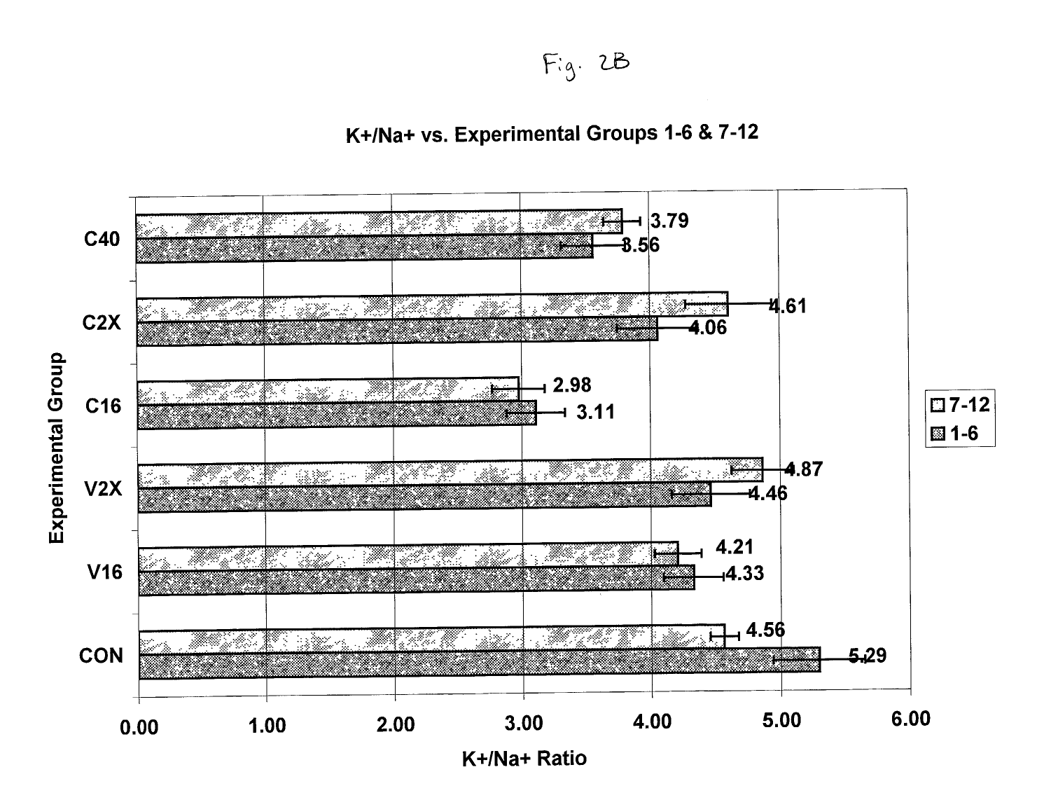

This experiment was followed up by determining concentrations of cryoprotectant that would vitrify in the presence of a 2× vehicle solution. Furthermore, the issue of chilling injury was investigated. Past published literature has indicated the chilling injury in kidney is probably due to cell shrinkage caused by incomplete penetration of cryoprotective agents into renal cells. Therefore, exposing slices to a vitrification solution in a 2× vehicle solution prior to abrupt cooling to −20° C. was expected to lead to worse chilling injury than cooling slices to the same temperature in the same way but in the presence of a 1× vehicle solution. In fact, the exact opposite was observed. The results are given in FIG. 2 and Table 2 (on the same page in this disclosure.)

In FIG. 2, slices were exposed to two vitrification solutions or to vehicle solution only. “Con” refers to controls exposed only to RPS-2 at 0 degrees C. V16 refers to exposure for 20 min at 0 degrees C to a vitrification solution known as “V16”, a solution which is particularly advantageous in view of its very high stability against freezing on cooling and on warming and in view of its low toxicity. V16 is the same as 55% w/v Veg solutes in 1× RPS-2, except that 2% DMSO is added to the formula and 2% formamide is subtracted. “V2×” refers to exposure to a solution containing 52% w/v Veg solutes (DMSO, formamide, and ethylene glycol, in the standard proportions) in a 2× RPS-2 vehicle solution, and, as usual, at 0 degrees C for 20 min. the group “C16” refers to cooling abruptly to −20 degrees C by transferring slices from V16 at zero degrees C (after 20 min at that temperature) to V16 at −20 degrees C, and subsequent holding at −20 degrees for 20 min. The group “C2×” was treated in an identical fashion

| | | 10% D | 10% V | to 40% D | to 40% V | to 20% V | 20% to 40% |
|---|---|---|---|---|---|---|---|
| 1-6 | mean | 6.95 | 7.02 | 2.26 | 7.16 | 6.61 | 6.49 |
| | variance | 0.13 | 0.27 | 0.13 | 0.16 | 0.38 | 0.29 |
| | stdev | 0.36 | 0.52 | 0.37 | 0.40 | 0.62 | 0.54 |
| | stderror | 0.15 | 0.21 | 0.15 | 0.18 | 0.25 | 0.22 |
| | % control | 100.00 | 101.10 | 32.56 | 103.04 | 95.15 | 93.47 |
| | sum | 41.68 | 42.14 | 13.57 | 35.79 | 39.66 | 38.96 |
| | n | 6 | 6 | 6 | 5 | 6 | 6 |
| 7-12 | mean | 6.79 | 6.50 | 2.28 | 6.63 | 6.30 | 6.38 |
| | variance | 0.15 | 0.25 | 0.13 | 0.32 | 0.04 | 0.47 |
| | stdev | 0.38 | 0.50 | 0.36 | 0.57 | 0.20 | 0.69 |
| | stderror | 0.16 | 0.20 | 0.15 | 0.23 | 0.08 | 0.28 |
| | % control | 100.00 | 95.80 | 33.55 | 97.64 | 92.76 | 93.93 |
| | sum | 40.72 | 39.01 | 13.66 | 39.76 | 37.77 | 38.25 |
| | n | 6 | 6 | 6 | 6 | 6 | 6 |

| | | 55% w/v Veg | | 55% w/v Veg in | | 40% then 52% w/v |
|---|---|---|---|---|---|---|
| | Control, | in 1X RPS-2 + | 52% w/v Veg in | 1X RPS-2 + | 52% w/v Veg in | Veg in 2X RPS-2 |
| | 1X RPS-2 | 2% DMSO | 2X RPS-2 | 2% DMSO @ −20 | 2X RPS-2 @ −20C. | @ −20C. |
| | CON | V16 | V2X | C16 | C2X | C40 |
| 1-6 | 5.42 | 4.43 | 4.57 | 3.31 | 4.08 | 2.9 |
| | 5.47 | 4.59 | 5.05 | 2.71 | 4.52 | 3.06 |
| | 6.8 | 5.18 | 5.07 | 2.31 | 4.08 | 3.54 |
| | 4.27 | 3.7 | 4.73 | 3.94 | 3.06 | 4.33 |
| | 5.1 | 4.38 | 3.07 | 3.15 | 5.2 | 4.27 |
| | 4.7 | 3.68 | 4.29 | 3.24 | 3.4 | 3.28 |
| 7-12 | 4.59 | 4.03 | 4.55 | 2.83 | 5.32 | 4.27 |
| | 4.7 | 4.96 | 4.12 | 3.68 | 4.8 | 3.62 |
| | 4.72 | 3.67 | 4.52 | 3.1 | 4.3 | 3.87 |
| | 4.32 | 3.91 | 5.59 | 2.57 | 5.38 | 3.72 |
| | 4.88 | 4.27 | 5.52 | 3.34 | 4.68 | 4.02 |
| | 4.17 | 4.4 | 4.89 | 2.33 | 3.17 | 3.24 |
| mean | 4.928333 | 4.266667 | 4.664167 | 3.0425 | 4.3325 | 3.676667 |
| variance | 0.519161 | 0.243752 | 0.452608 | 0.259002 | 0.651657 | 0.240133 |
| stdev | 0.720528 | 0.493712 | 0.672762 | 0.508923 | 0.807253 | 0.490034 |
| stderror | 0.207999 | 0.142522 | 0.19421 | 0.146913 | 0.233034 | 0.141461 |
| % control | 100 | 86.57423 | 94.63984 | 61.73487 | 87.91004 | 74.60264 |
| sum | 59.14 | 51.2 | 55.97 | 36.51 | 51.99 | 44.12 |
| n | 12 | 12 | 12 | 12 | 12 | 12 |

TABLE 2B

| | | | 55% w/v Veg in | | 55% w/v Veg in | | 40% then 52% w/v |
|---|---|---|---|---|---|---|---|
| | | Control, | 1X RPS-2 + | 52% w/v Veg | 1X RPS-2 + | 52% w/v Veg | Veg in 2X |
| | | 1X RPS-2 | 2% DMSO | in 2X RPS-2 | 2% DMSO @ −20 | in 2X RPS-2 @ −20C. | RPS-2 @ −20C. |
| | | CON | V16 | V2X | C16 | C2X | C40 |
| 1-6 | mean | 5.293333 | 4.326667 | 4.463333 | 3.11 | 4.056667 | 3.563333 |
| | variance | 0.750787 | 0.324387 | 0.553227 | 0.30948 | 0.589507 | 0.372267 |
| | stdev | 0.866479 | 0.56955 | 0.743792 | 0.556309 | 0.767793 | 0.610137 |
| | stderror | 0.353739 | 0.232518 | 0.303652 | 0.227112 | 0.31345 | 0.249087 |
| | % control | 100 | 81.73804 | 84.3199 | 58.75315 | 76.637286 | 7.31738 |
| | sum | 31.76 | 25.96 | 26.78 | 18.66 | 24.34 | 21.38 |
| | n | 6 | 6 | 6 | 6 | 6 | 6 |
| 7-12 | mean | 4.563333 | 4.206667 | 4.865 | 2.975 | 4.608333 | 3.79 |
| | variance | 0.071627 | 0.203227 | 0.34571 | 0.24939 | 0.661537 | 0.1252 |
| | stdev | 0.267632 | 0.450807 | 0.587971 | 0.49939 | 0.813349 | 0.353836 |
| | stderror | 0.10926 | 0.184041 | 0.240038 | 0.203875 | 0.332048 | 0.144453 |
| | % control | 100 | 92.18408 | 106.61076 | 5.19357 | 100.9861 | 71.5995 |
| | sum | 27.38 | 25.24 | 29.19 | 17.85 | 27.65 | 22.74 |
| | n | 6 | 6 | 6 | 6 | 6 | 6 |

except the cryoprotectant solution was the “V2×” solution instead of the “V16” solution. Finally, the last group involved a hybrid experiment between the best method known in the prior art for avoiding chilling injury and the current use of a hypertonic (greater than 1×) vehicle solution. In this group, slices were exposed to only 40% Veg at 0 degrees C for 20 min, in 1× RPS-2, and were then transferred to the V2× solution precoooled to −20 degrees C and held for 20 min at that temperature as in the other chilled groups.

The results, as indicated in the figure, are extraordinary. First, as noted above, the V16 group did very well, giving an average K/Na ratio of 4.22 against the control ratio of 4.83, which represents recovery of 87% of control viability (see tabular documentation as well). However, the V2× group did even better, reaching 96% of control viability (third bar from the bottom, K/Na=4.64). Cooling slices in V16 to −20 led to the expected drop in viability (K/Na of 3.05, representing just 63% of control viability). But cooling slices in V2× to −20 yielded a K/Na ratio of 4.34, or 89.9% of control viability. Finally, using prior art technology for circumventing chilling injury, namely, cooling slices in a lower concentration of cryoprotectant to avoid cell shrinkage mediated chilling injury, but cooling them by immersing them in V2×, which is hypertonic (over 1× vehicle solution concentration), resulted in a K/Na about halfway between the results of cooling in 1× vehicle (the C16 group) and the results of cooling in 2× vehicle (the C2× group). Therefore, it was detrimental to cool from 40%, 1× as compared to cooling from 52%, 2×, in complete contradiction to the prior art, yet it was beneficial to cool into a 2× solution even starting from the initial 40%, 1× solution, a opposed to cooling into a 1× solution, again in contradiction to the prior art. Therefore, the data support, in multiple ways, the new invention, which includes:

The use of hypertonic medium to reduce both cryoprotectant toxicity and chilling injury, wherein the hypertonic medium is added and removed in a manner that simulates the effects of both freezing an thawing, and

The use of a hypertonic medium to reduce the time required to remove cryoprotectant from living cells, wherein the accelerated washout of cryoprotectant is achieved by simulating thawing (simultaneous dilution of the hypertonic medium and the cryoprotectant, a strategy that is wholly lacking in the prior art), and

The use of a hypertonic medium to protect living cells from dilution shock, wherein the protection is achieved by the presence of the hypertonic medium prior to dilution and maintained by simultaneous dilution of the cryoprotectant and the medium in at least roughly similar proportions.

What is claimed is:

1. A method for cooling and rewarming living cells with lower chilling injury and cryotoxicity to a living cell, comprising:
adding a nontoxic concentration of cryoprotectant to said cell in a physiologically supportive media;
increasing the cryoprotectant and physiologically supportive medium in the same proportion to achieve the desired concentration of cryoprotectant;
cooling to the desired temperature with greatly reduced toxicity and chilling injury;
warming;
returning the concentration of cryoprotectant and supportive medium to a lower concentration.

2. The method of claim 1 wherein said lower concentration is the initial concentration.
